(19) United States
(12) Patent Application Publication
(10) Pub. No.: US 2019/0021616 A1
(43) Pub. Date: Jan. 24, 2019

(54) Body Worn Biometrics Assembly and Method of Operating Same

(71) Applicants: Lawrence J. Day, Grand Blanc, MI (US); Michael L. Bottom, Ann Arbor, MI (US)

(72) Inventors: Lawrence J. Day, Grand Blanc, MI (US); Michael L. Bottom, Ann Arbor, MI (US)

(21) Appl. No.: 15/861,615

(22) Filed: Jan. 3, 2018

Related U.S. Application Data
(60) Provisional application No. 62/441,752, filed on Jan. 3, 2017.

(51) Int. Cl. A61B 5/024; A61B 5/107

(52) U.S. Cl. CPC A61B 5/02427; A61B 5/6802; A61B 5/1079

(57) ABSTRACT

A biometrics device measures a biometric quantity of a body of a user while the user is in motion. The biometric device includes a sensor to measure the biometric quantity of the user and creates a sensed biometric signal. A coupling device couples the sensor to the body of the user. An emitter is electrically connected to the sensor, receives the sensed biometric signal, and emits an output signal to be received by the user such that the user can understand the biometric quantity as the user continues the motion.

This patent application claims priority to a U.S. patent application having application Ser. No. 62/441,752, filed on Jan. 3, 2017, the disclosure of which is incorporated herein by reference.

### BACKGROUND ART

### 1. Field of the Invention

The invention relates to accurate sensing and real-time display of biometrics. More particularly, the invention relates to real-time display of biometrics while the wearer is active.

### 2. Description of the Related Art

There are multiple problems with the current state of the art. As stated, lack of an easily readable waterproof display for swimmers and other exercisers to conveniently view accurate data, in real-time, during activity without altering stroke mechanics or exercise movements is a major disadvantage. Pulse rate data from currently available, popular wrist wearables is not accurate for people in motion. “[Errors] ranged from +/−34 beats per minute to +/−15 beats per minute, depending on the type of activity.” (American College of Cardiology, Marc Gillinov, MD, Cleveland Clinic, Mar. 8, 2017).

Commercially available devices include chest strap, finger-tip-hinged clamshell pulse oximeters, wristwatch, wrist band, smartphone, smartwatch, ear lobe clip biometric sensing devices. All of these devices are either inaccurate, uncomfortable, not submersible, and/or provide inconsistent data. The outputs of these devices are difficult to view in real-time during activity by both the wearer and the coach. These devices also present unacceptable form factors and frequently come loose during swimming and other exercise movements in or out of the water. All devices available to the general public are imprecise and inconsistent in measuring and conveniently displaying real-time heart rate and/or other key biometrics during activity. The reasons for this include, but are not limited to, over-reliance on light sensor technologies (infrared and/or other forms) and/or lack of a fixed, secure, close, intimate interface with the skin to facilitate high quality sensor readings at advantageous biometric measuring and display viewing sites on the body for a given exercise. Moreover, skin perfusion (how blood flows through skin) is another factor that may distort accuracy of light sensor based heart rate monitors. Skin perfusion differs widely between people and is affected by internal conditions, including, but not limited to, thickness of skin, pigmentation of skin, depth of blood vessels. External conditions (for example, without limitation, ambient lighting, air and/or water temperature if swimming, etc.) may also affect the skin, skin perfusion and light sensor-based heart rate readings. Attempting achieve closer skin-sensor contact by cinching down on a wristwatch-type heart rate monitoring device can alter skin perfusion impacting heart rate sensor accuracy. In essence, light sensors need to “see” through the skin into the blood vessels. Anything that alters the light sensor “view” through the skin can distort the accuracy of sensor readings. These light sensor accuracy issues plague heart rate monitor wearables (especially wrist wearables) currently available in the marketplace. Solutions to these problems are provided in multiple iterations of the proposed invention.

Most, if not all, heart rate activity monitoring devices available to the general public rely on photoplethysmography (light signal sensors). Readings from light-based sensors may be inconsistent and inaccurate during activity in currently available wearables for reasons including, but not limited to lack of a fixed, secure, appropriately close, intimate, uninterrupted contact connection between the skin and sensors at advantageous body data monitoring and display locations. These devices, to be effective during activity, among other things, must sustain a secure contact and interface at the biometric data harvesting locations on the body with the least amount of ambient “noise” in a given exercise environment. By way of example, a wristwatch device on a freestyle swimmer will encounter the noise, turbulence and possible disruption of the skin-sensor connection, caused by hands “crashing” through the water surface on every stroke. Availability of good options for sensor site placement on the body, depending on the nature of a given exercise, greatly impacts accuracy of readings and conspicuous, real-time, display back to the user and others. Wrist, ear lobe, chest and other devices dedicated to use on only one location of the body lack this important positional flexibility. By contrast, the preferable, but not exclusive, forms of the proposed invention, integrated into adhesive epidermal patches and/or skin strips and/or gear and/or compressive, elastomer based, non-adhesive gear may open the way to access many different body-sensor and display sites well beyond those traditionally used. The possibility of an expanded array of more accurate biometric data sensing sites and easily viewed display locations is facilitated by the close, stable, unchanging, secure, high quality skin contact provided by flexible, adhesive epidermal strips/patches and/or non-adhesive and/or compressive gear as described herein.

Research on heart rate monitors demonstrates there is heavy reliance on light sensor technology (photoplethysmography) for devices available to the general public. Regrettably, as presently deployed, it appears that light based sensing technologies built into wearables are only accurate while the wearer is almost motionless. (American College of Cardiology, cited above). Photoplethysmography or other, possibly more precise biometric sensing technologies, may be facilitated and/or improved when a close, appropriately fixed, high fidelity connection between sensors and skin is established as described herein. Some sensor technologies, other than photoplethysmography, include, but are not limited to electrical impulse (electrocardiography), and/or pressure sensing (oscillometric pulses) and/or sound sensing (phonocardiograph), chemical sensors, quantum sensors, diamond nanocrystal sensors, nanotube sensors, nanotechnology based sensors, sensors wherein optical fibers are embedded in composite materials (where the material, itself, essentially acts as a sensor), nanotube sensors, and others.

Minimal use of non-light based biometric sensing modalities during activity and inaccurate photoplethysmography (light sensor) readings may, at least in part, be due to failure to secure and maintain a close, intimate, highly communicative relationship to and from the inner body, through the skin, and into sensors. A seamless, optimally close contact, uninterrupted “bridge” or “link” between the epidermis and sensors is needed to facilitate accurate, high quality transmission of biometric data from the inner body to external monitoring devices.

One of the embodiments is briefly mentioned here because it directly addresses present day problems with heart rate monitor wearables commercially available in the marketplace that fail to sustain an optimally intimate sensor connection with the skin.

Currently available chest strap devices attempt to harvest biometric data from the advantageous-mid-chest sensing area. Regrettably, chest straps frequently fall out of position, lose close connection with the skin or allow water or perspiration to interfere with the fidelity of sensor readings. Moreover, chest strap monitors typically attempt to transmit data wirelessly, via radio frequency (RF), viewable on wristwatch type displays that are small and difficult to view in real-time by swimmers and other exercisers while in motion. Compounding the problem, is the fact that wireless RF transmissions may rapidly attenuate in water.

In summary, all commercial body-worn devices available to the general public (i.e. chest straps, finger-tip, hinged clamshell pulse oximeters, wristwatches, wrist bands, ear lobe clip devices, smartphones, smartwatches, etc.) that claim to measure heart rate and other biometrics of people during activity may have some or all of the following disadvantages:

Displays worn at locations on the body that are difficult for swimmers, exercisers to view and interpret without altering stroke, stride, dance or other exercise movements;

Small displays difficult to see while swimming and/or exercising;

Displays that cannot be directly viewed in real-time by coaches, trainers and observers of exercisers while in motion;

Inaccurate and inconsistent readings;

Bulky form factors for exercisers, including but not limited to swimmers who want to be sleek and streamlined in the water;

Rely on photoplethysmography (light sensing technology);

Lack a close, secure, fixed, intimate, connection, interface and contact of sensors to the epidermis resulting in inaccurate transmission of biometric data through the skin to sensors. This poor contact also inhibits use of other biometric sensing technologies, including, but not limited to, electrical impulse, and/or pressure sensing and/or sound sensing, deep tissue sensors and/or deep tissue laser sensors, etc.

Are designed to be worn or attached only at certain dedicated locations on the body. This limits options for advantageous positioning of devices for optimal accuracy of biometric sensor readings in various activities. It also limits options for direct viewing, by the wearer and others, of optical output and/or for viewing in reflective surfaces as described herein. Limited display locations on the body may limit options for positioning detached reflective surfaces (i.e. mirrors or holograms, etc.) for ease of viewing by the wearer and observers, depending on multiple factors including, but not limited to, type of exercise, anatomy of user, skin perfusion rate and environment;

Are designed to be worn or attached with sensors only at certain locations on the body thus impeding the ability to use other heart rate and biometric sensing technologies that may require particular body-attachment sites and/or require two or more contact locations on the body, including, but not limited to ECG/EKG electrode placements;

Attempt to wirelessly transmit heart rate from a sensing-advantageous location, such as the chest, to a wrist display location (for example and not by way of limitation) using radio frequency waves that rapidly attenuate in water. (Note: An embodiment of the invention, below describes use of sensor data from the chest transmitted using near field magnetic communication, “NFC,” to a separate display and/or displays at different locations on the wearer's body.). Unlike RF, NFC travels equally well in air and water.

Need to be recharged frequently due to power hungry photoplethysmography sensors, capacitive screens and/or multiple function devices;

Have capacitive touch screens that can be problematic for activities in water;

Other disadvantages that may be discovered during the pendency of this APPLICATION and/or described in subsequent filings with the USPTO.

Example embodiments of the proposed invention addressing the above stated problems are further discussed below. Embodiments could include the invention in many adhesive and/or non-adhesive and/or compressive iterations. Where embodiments are described as “preferred” or “preferable” it should not be construed as precluding other possibilities. As sensor technology evolves differing embodiments may become preferred iterations.

### SUMMARY OF THE INVENTION

The primary objectives of the proposed invention are to provide real-time, accurate biometric feedback, including but not limited to heart rate, conveniently and simultaneously viewable by wearers of the device, while in motion, and observers such as coaches.

The proposed invention seeks to facilitate all of the following goals:
Accurate, closed-loop, bio-metric feedback including but not limited to heart rate;
Delivered in real-time;
During activity;
In devices with streamline form factors;
With easily viewed data conveniently displayed simultaneously to athlete and coach (wearer and observer);
Without disruption of the wearer's exercise movements;
Optionally submersible and/or water resistant and/or waterproof.

Comprehensive attainment of all these goals in one device, apparatus, method and/or system has eluded industry for decades and continues to do so.

Many exercisers, athletes and coaches use heart rate as a key training guide. People engaged in activities, including exercisers, athletes and their coaches frequently want to view biometrics, including heart rate, in real-time, while in motion. This closed loop feedback is critical to their training. However, displays showing optical output from-body worn and/or attached heart rate monitors can be difficult to see while in motion because the displays may be inconveniently positioned on the body and/or too small. Co-inventor of the proposed invention, 2016 USA Olympic Swimming Coach, Mike Bottom, knows this problem is especially true for competitive swimmers. These athletes and their coaches want accurate heart rate and other data during active training. They also want proper stroke mechanics and streamlining to be maintained at all times while the athlete is moving through the water. Currently available heart rate monitor wearables are inaccurate during activity and/or have unacceptable form factors or other problems discussed below.

People, during exercise, including but not limited to swimmers and coaches observing them, would benefit from a conveniently readable optical display of biometric data from comfortable, waterproof, body-attached and/or worn devices, conspicuously showing high fidelity heart rate and other biometrics to them in real-time, without altering exercise movements. Such optical data could be displayed alphanumerically and/or non-alphanumerically (i.e. including but not limited to color coded and/or blinking lights). Relevant data might include, but is not limited to, heart rate, blood pressure, blood oximetry, body temperature, heart rate variability, ambient temperature, respiration rate, oxygen perfusion, skin and muscle tension, lactic acid levels, blood chemistry, elapsed time, elapsed time per distance traveled, lengths, laps, stroke rate, step rate, stride rate, number of strokes or steps, strokes per length, steps or strides per lap, accelerometer data and other information.

Applicants know of no invention and/or device and/or system, commercially available to the public, that accurately accomplishes all the above mentioned goals.

Embodiments of the proposed invention would be capable of achieving all of the goals and addressing the problems mentioned above. Iterations include, without limitation, devices, apparatus and/or systems and/or methods having one or more sensors capable of accurately sensing biometrics and other data. Said data is immediately transmitted to integrated and/or connected (via wire and/or wirelessly) displays, capable of showing non-alphanumeric optical output (including, but not limited to, color-coded lighting and/or blinking-lights). Optical output may also be in alphanumeric form (including, but not limited to digits, letters, signs, etc.) and capable of regular and/or laterally inverted display. Sensors and displays, together with other supportive electronics mentioned below, are integrated into or connected to, optionally waterproof and submersible, programmable, non-adhesive (elastically compressed to the skin) and/or adhesive body-attached or worn gear and/or patches and/or strips having a wide degree of flexibility, ranging from pliable to rigid. Generally, all such devices would be attached to or worn by a person allowing the wearer and others to simultaneously view digital, alphanumeric and/or non-alphanumeric (i.e., without limitation, color coded or blinking light coded) data, such as heart rate and other biometrics and metrics in real-time. Views by wearers and observers could be either direct and/or in detached reflective surfaces (i.e. without limitation, polished aluminum, mirrors and/or holograms). Views of laterally inverted alphanumeric displayed data from mirrors would be properly oriented upon reflection and seen in real-time, during active exercise. This iteration of the invention could also be used in combination with normal displays of data (not laterally inverted) facilitating simultaneous direct viewing of optical output by others, including coaches and trainers (for example, including, but not limited to, instructors leading stationary cycling classes). There are multiple embodiments, configurations, applications and uses of the invention. All embodiments are intended to address all of the goals and problems discussed above.

The embodiments disclosed herein are illustrative and not intended to limit other iterations and/or designs and/or possible constituent and/or affiliated components of the proposed invention in any way (whether deemed a device, apparatus and/or system and/or method). Other embodiments may include additional elements and/or supportive devices, components, apparatuses, systems, methods, and/or applications and/or uses, programmability, hardware, software, technologies, material technologies (for example, without limitation, conductive polymers), sensing technologies and optics technologies (i.e., such as, without limitation, magnifiers, amplifiers, prisms, beam splitters, near field magnetic communication, “NFC,” and others mentioned or not mentioned in this APPLICATION, etc.).

Generally, but not in every instance, preferred embodiments of the proposed invention will include, but are not limited to, displays enabled to show regular and/or laterally inverted alphanumerical optical output and/or non-alphanumeric optical data (including, but not limited to colors and/or blinking lights). These displays are integrated into and/or used in conjunction with fully equipped and purpose-adapted biometrics and/or data monitors. The proposed invention, device and/or apparatus and/or system and/or method may also include or work with many other elements or components, including, but not limited to, flexible and/or rigid plastic/polymer display screens, flexible and/or rigid organic light-emitting diodes in flexible and/or rigid, and/or curvilinear plastic/polymer screens (optionally waterproof), sensors, multi-detectors, light emitting diodes (LEDs), organic light emitting diodes (OLEDs), liquid crystal displays, conductive and/or non-conductive polymers, micro-controllers, processors, memory storage, integrated chips, applied specific integrated chips (ASICs), switches, electronics, supportive circuitry, hardware and software, reflective surfaces (i.e. without limitation, mirrors or polished metal of various shapes including planar, banana and/or holograms, etc.), optics, enhanced eyewear (i.e. without limitation, goggles, masks, glasses and eye contacts), batteries (with traditional and/or inductive charging systems), solar panels and other components. These components and others may or may not be integrated into comfortable, optionally waterproof, submersible adhesive or non-adhesive (for example, but without limitation, elastomers with compressive forces) gear and/or skin/epidermal patches and/or strips of various shapes and sizes to facilitate optimal skin contact, accurate sensing, optimal form factor and convenient real-time viewing of accurate biometric data by wearers and observers. Embodiments of the invention may use or work with a variety of sensor, display and/or wireless communication technologies. Said adhesive and/or non-adhesive gear and/or epidermal patches and/or skin strips may be flexible or rigid, thin, optionally waterproof, submersible, employing non-adhesive elastic compressive forces (and/or other methods), and/or adhesive technologies, all of which provide an intimate connection and interface between the skin and sensors for highly accurate readings of biometrics and other data from a variety of body sites.

Alphanumeric, optical output of normally displayed data on displays integrated into and/or coupled with and/or in communication with sensors could be understood when directly viewed. Laterally inverted alphanumerically displayed optical data could be relayed, and properly oriented in the mirror image, using detached mirrors (reflective surfaces), back to the wearer, in real-time. As stated, direct views of normally displayed data could also be available to the wearer and/or to observers (i.e. coaches) depending on location of displays. Non-alphanumeric data (including, but not limited to colors and blinking lights) would not require laterally inverted display for direct viewing or viewing in mirrors.

### DESCRIPTION OF THE DRAWINGS

FIG. 1 is an above water view, looking down on a swimmer 1(a) over a mirror 1(b) on the pool bottom. Display from a round, adhesive chest patch monitor 1(c) with non-alphanumeric optical output (i.e. color coded or blinking light indications of programmed heart rate zones) is depicted in the mirror reflection.

FIG. 2 is an above water view, looking down on a swimmer 2(a) over mirror 2(b) on pool bottom. Display from a rectangular, adhesive chest patch monitor 2(c) with horizontal, laterally inverted, alphanumeric optical output, is depicted as being reflected, in proper orientation, in the mirror image.

FIG. 3 is a view of the underside of the wrist and hand 3(a) showing through a swimmer's hand paddle 3(b). Laterally inverted numbers 3(c), representing heart rate, are shown on the hand paddle center display 3(d). An adhesive oval monitoring sensor patch 3(e) is depicted on the bottom side of the wrist over the area of the radial pulse. Although sensor data could be transmitted wirelessly, this embodiment employs a wire running from the sensor patch 3(f) for transmission of alphanumeric optical output to the center display 3(d). The center display 3(d) as well as the entire paddle 3(b) could also be used to display non-alphanumeric optical output (i.e. color coded or blinking light indications of programmed biometric zones).

FIG. 4 is a view of the underside of the wrist and hand 4(a) showing an adhesive oval monitoring sensor patch 4(b) with EKG-type male snap fastener connector 4(c) on the wrist over the area of the radial pulse.

FIG. 5 is a non-adhesive wrist bracelet or band for color-coded or blinking-light display of optical output depicted with attached EKG-type female snap fastener connector 5(a) that couples with the male snap fastener 4(c) of FIG. 4 on the same drawing page.

FIG. 6 is a view of the underside of the hand and wrist 6(a) with an adhesive embodiment of the device 6(b) wrapped all the way around the wrist. The device 6(b) is fully enabled to sense, interpret and display non-alphanumeric (color coded or blinking light data), and, optionally, could be programmed to display alphanumeric data.

FIG. 7 is an underwater view of a swimmer 7(a). Non-alphanumeric (color coded or blinking light data) display of optical data is viewable by wearer and observer on a fully enabled, oval, adhesive mounted sensor monitoring patch 7(b) located on the inside of the left elbow, over the brachial artery.

FIG. 8 is a view showing a double-sided adhesive, round patch (“sensor-bridge” embodiment) 8(a), depicted as slightly raised or peeled back on a wristwatch type wearable monitoring device 8(b). This “sensor bridge” provides a fixed, stable skin-to-sensor connection. This stable connection enhances fidelity of sensor readings on wristwatch type monitoring products 8(b), and similar products, commercially available in the marketplace even when the wearer is physically active. Also depicted are four circular components of a representative light sensing system 8(c). Only one of the four clustered circular components has an arrow pointing to it 8(c) to avoid crowding and confusion in the drawing. Typically, two of the four circular components 8(c) would be light emitters and two would be light-rebound-receiving-sensors. The double-sided-adhesive-patch (“sensor-bridge”) 8(a) could, optionally, be provided with and/or without openings where the light emitters and/or sensors are located. As stated herein, the “sensor-bridge” device could also include enhanced lenses, optics, polarizers, electronics, etc. to improve fidelity of light sensor readings. This “sensor-bridge” could also be employed with other sensor technologies.

FIGS. 9, 10, 11 comprise an exploded view showing a double-sided adhesive, round patch or disc 10 to improve sensor-to-skin connectivity in commercially available wearable wristwatch type biometric monitors. On top of the page is depicted an optional holding case frame 9 for this embodiment of the invention. Next, in the middle of the page, is the double-sided sensor-to-skin connecting patch 10. At the bottom of the page is depicted an example of a wearable wrist watch type device 11 with a cluster of four light sensor system components 11(a).

FIGS. 12, 13, 14, 15, 16, 17 provide views of six different possible embodiments of the invention employed in non-adhesive compression sleeves.

FIG. 18 is side and front view of a common swimming goggle or mask with silicone or silicone-like elastic, conformal material used for the frame, lens gasket and strap in which the entire invention could be contained. Seven light emitting diodes (LEDs) are shown within the goggle frame and/or gasket 18(a). These LED's can display non-alphanumeric optical output (i.e. blinking lights or color coded lights) visible, in real-time, to both swimmer and coach during activity.

FIG. 19 is a block diagram including laterally inverted display for mirror image viewing.

FIG. 20 is a block diagram for light sensors enhanced by a double-sided adhesive “sensor-bridge” embodiment of the invention.

FIG. 21 is a block diagram common to most embodiments.

### DESCRIPTION OF THE PREFERRED EMBODIMENTS

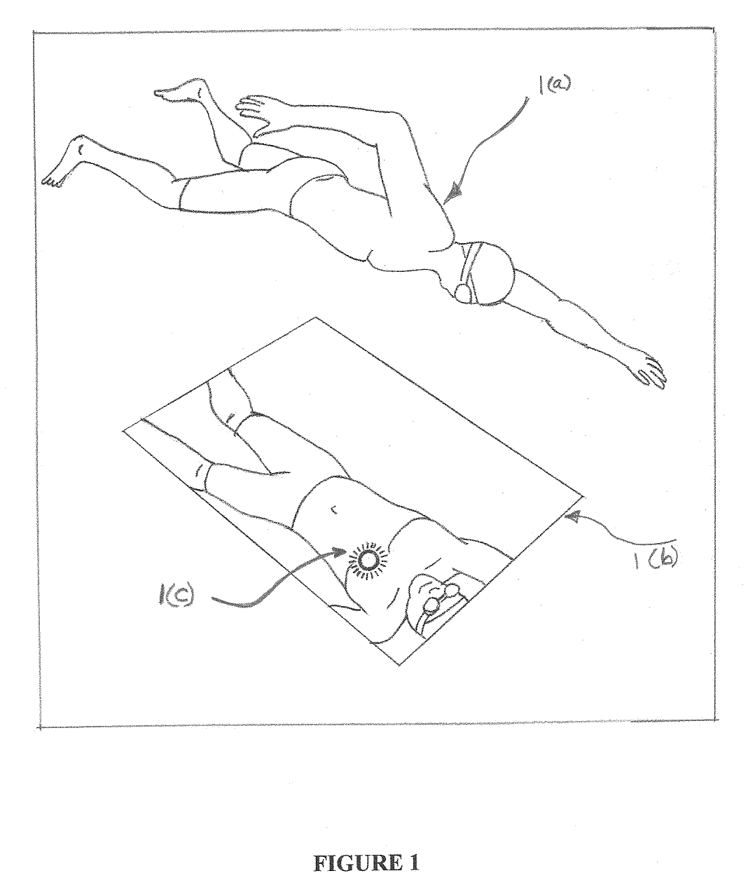

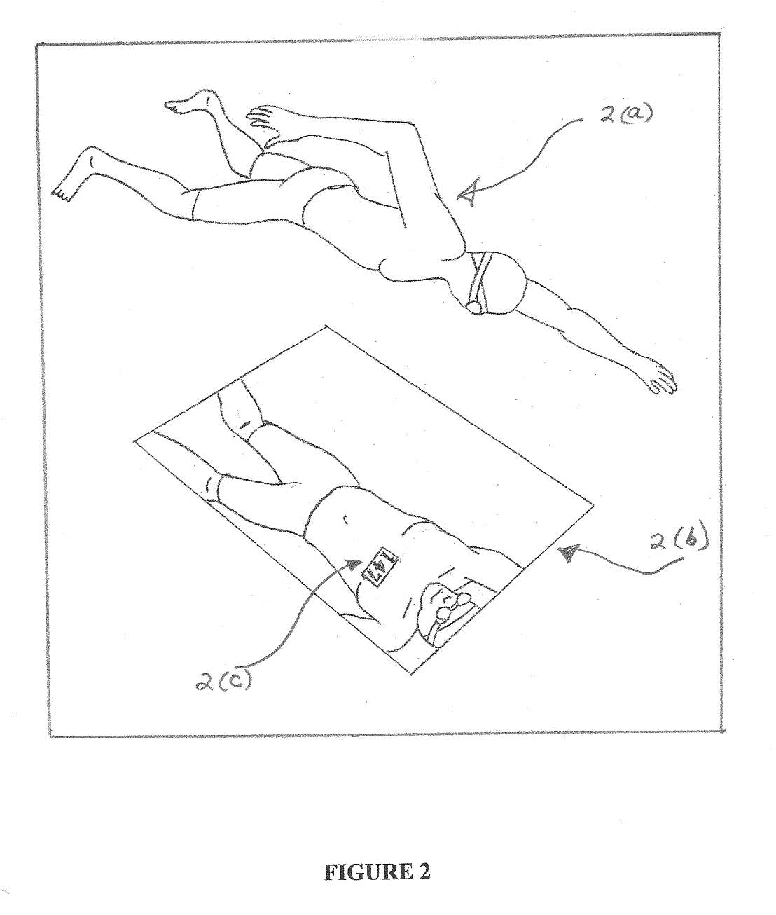

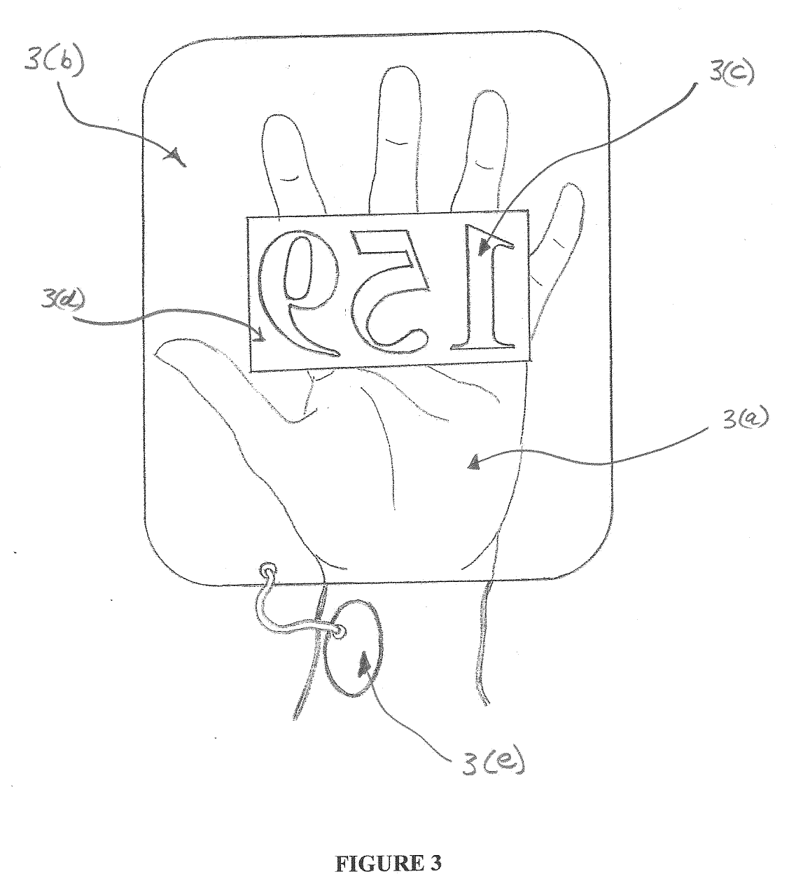

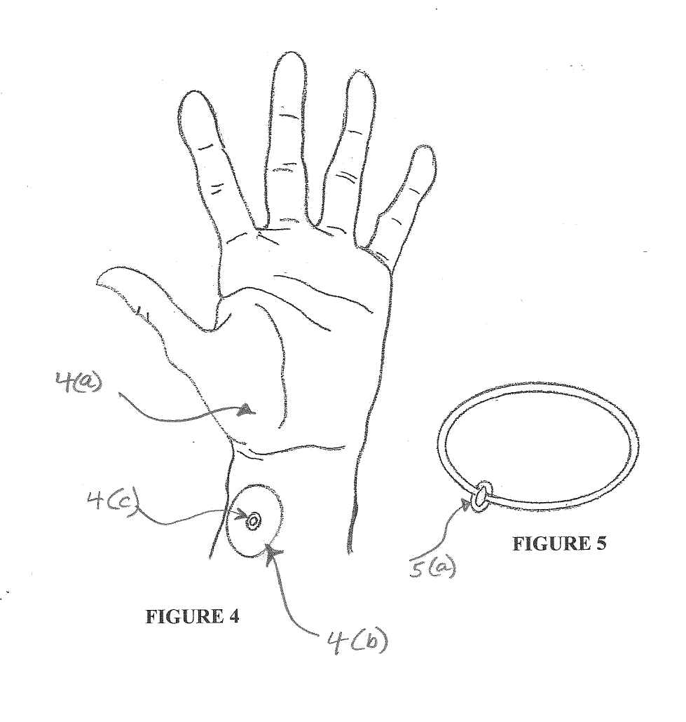

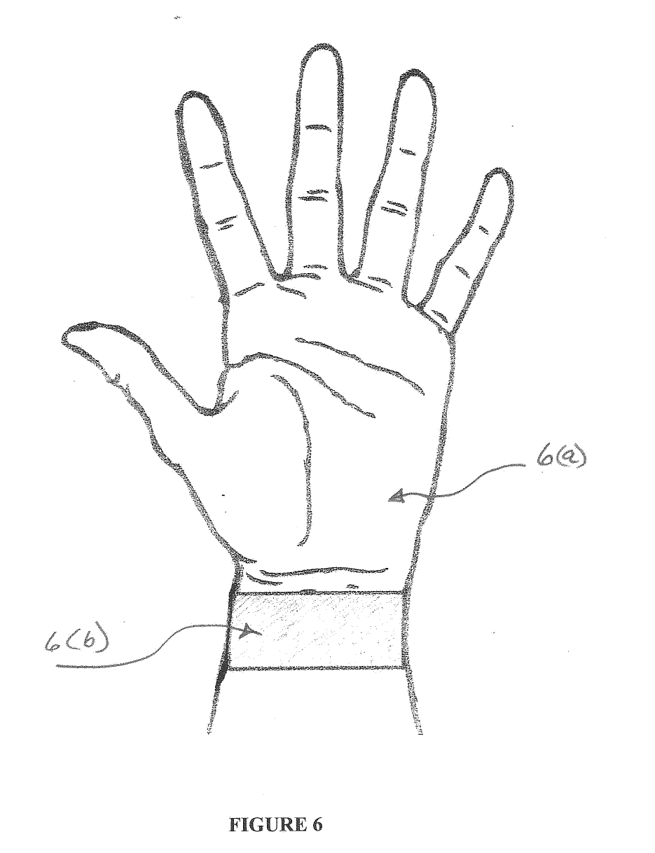

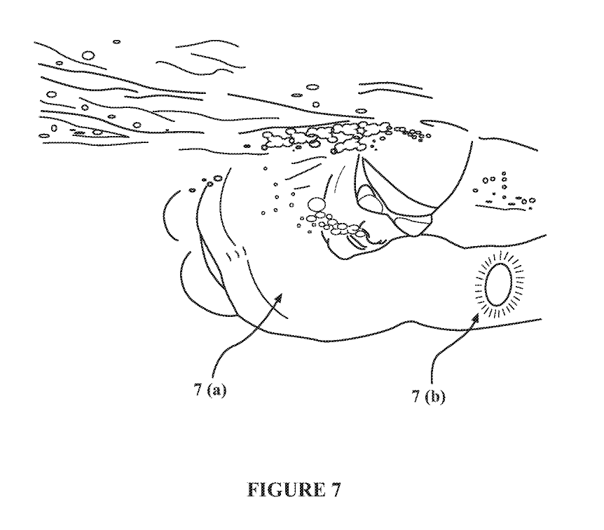

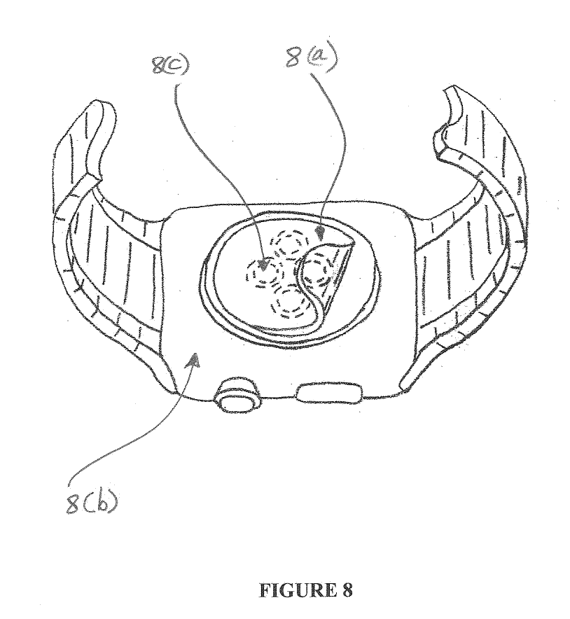

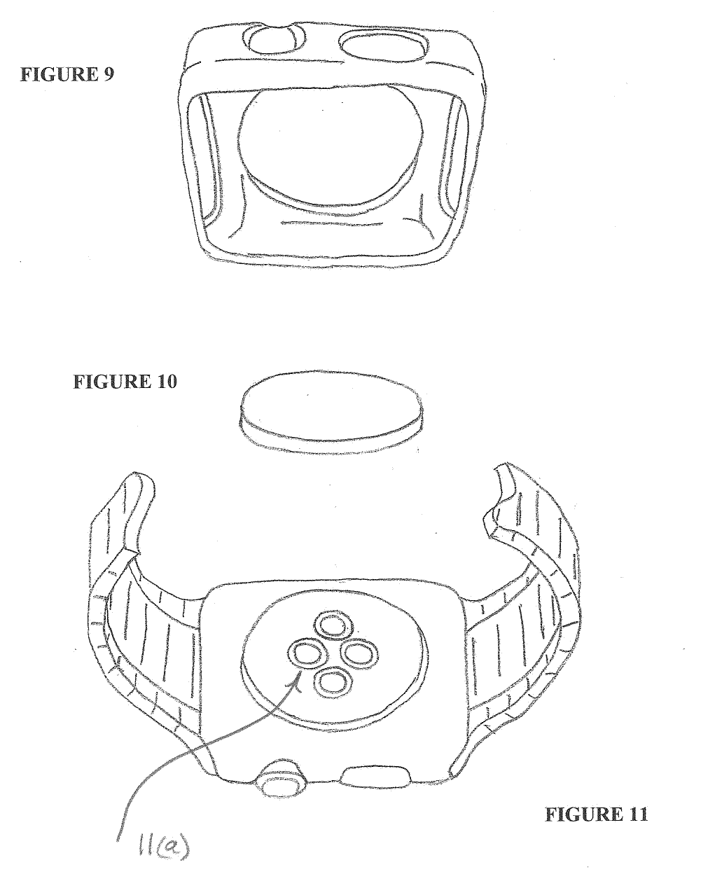

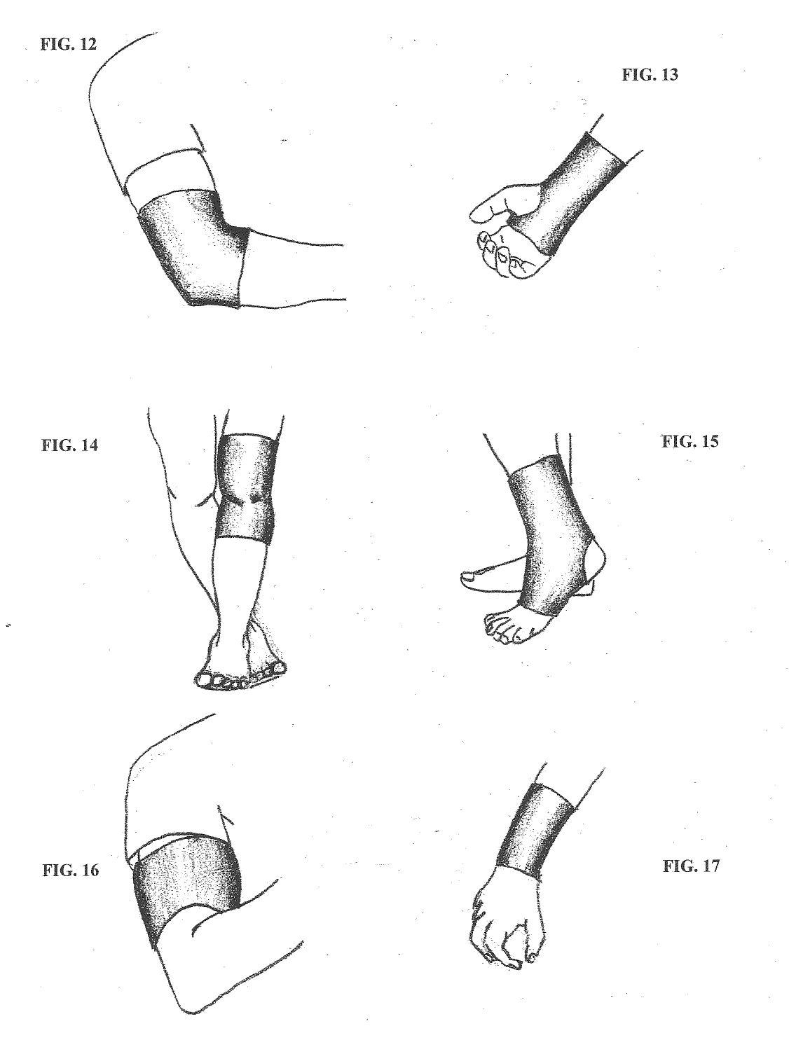

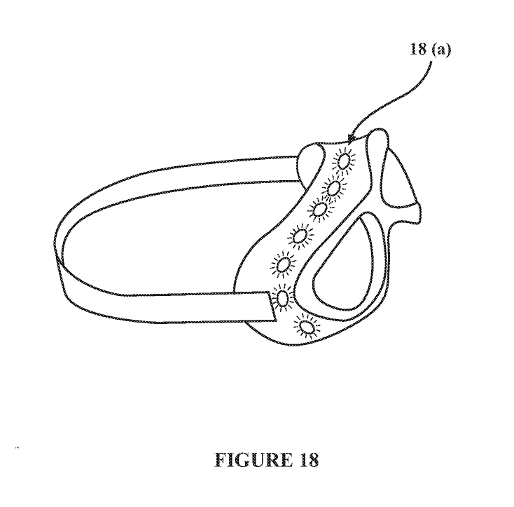

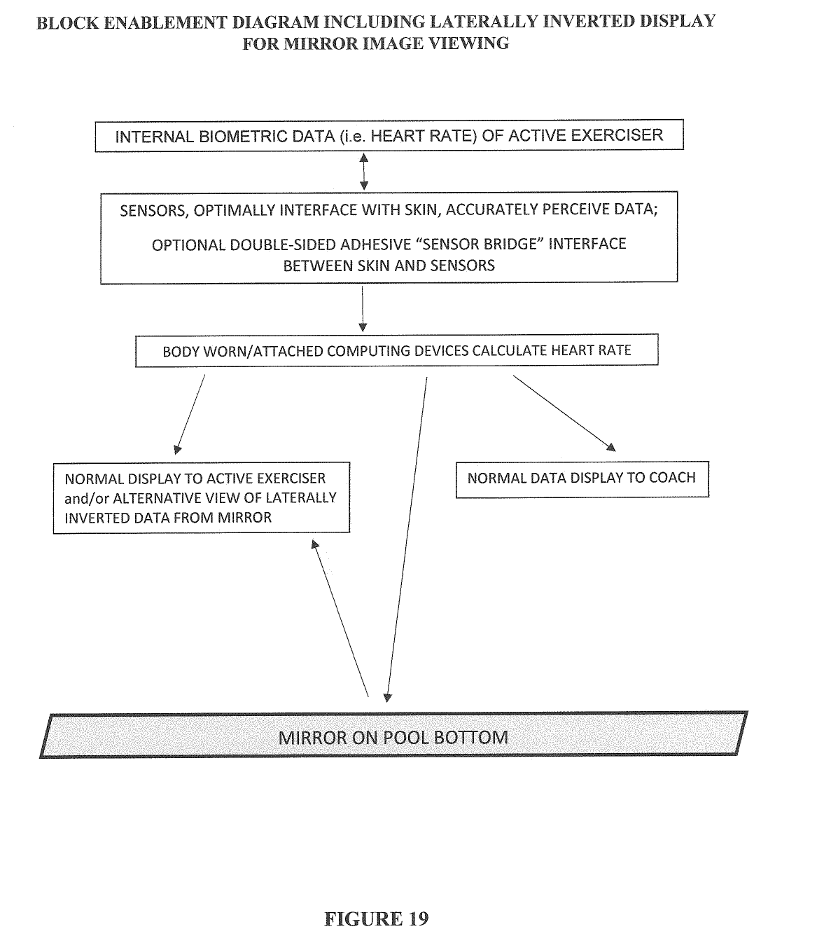

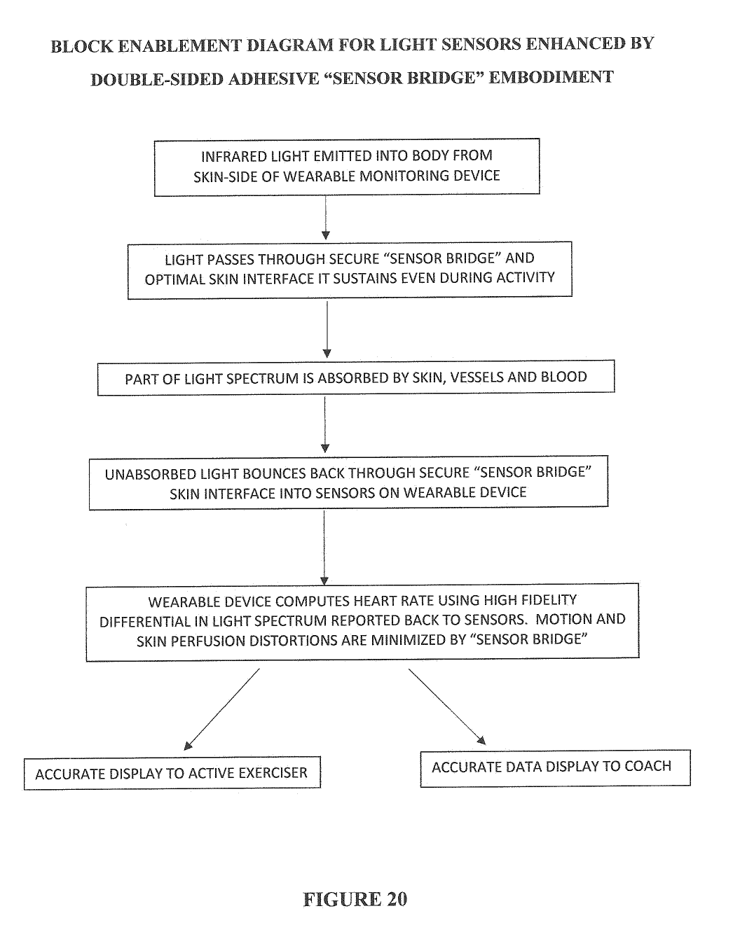

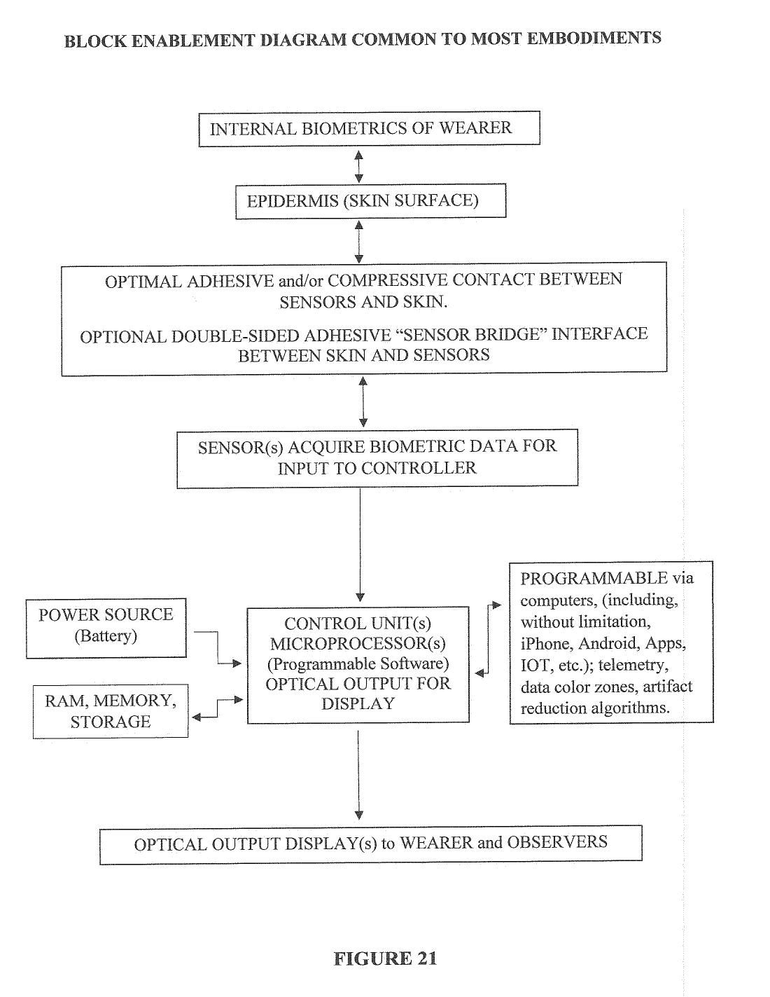

Referring to FIG. 1, one embodiment of the invention is attached to an upper extremity or other biometric sensing site visible to active athletes and their coaches in real-time: This embodiment or application of the proposed invention would benefit coaches and their athletes, including, but not limited to, swimmers. The device could be worn or attached on the arm, forearm, wrist, finger, inner elbow (i.e. inner elbow over the brachial, radial, ulnar arteries) or other advantageous biometric sensing sites visible to both athlete and coach and displaying either digital, alphanumeric and/or non-alphanumeric (for example, without limitation, color coded and/or blinking data). If attached and/or worn (i.e., including, but not limited to, in the form of an adhesive and/or non-adhesive and/or compressive skin strip, patch, gear, apparel on/over/around the elbow, upper arm, forearm, wrist, hand, fingers, etc.) on an upper extremity for example, data could be visible to a swimming coach on deck, above water, when a swimmer's arm came above the surface in the swimming stroke cycle. In one embodiment, the output of the device would be visible at least 50 meters away. The optical data output could also be visible to the swimmer directly and/or in mirrors, below the surface of the water, in real-time, when the attached device was underwater in the swimming stroke cycle

EMBODIMENT attached to or worn on the chest of a swimmer, visible to the wearer in underwater mirrors commonly used in flow-current flumes or pools (“aquatic treadmills”) as well as regular swimming pools, with optional additional device on another part of the body for simultaneous viewing by coaches:

This embodiment includes, but is not limited to, optionally waterproof, programmable, submersible, fully equipped and programmable, biometric monitoring devices, including, but not limited to, one or more integrated sensors and displays, capable of rendering laterally inverted optical output. It would be attached to and/or worn on the chest, benefiting from accuracy of heart rate and biometric sensing at that location. The chest is the gold standard for high fidelity human heart rate sensor readings. Preferential sensor location sites on the chest may vary depending on what heart related biometrics are sought. For example, some cardiologists prefer stethoscope placement at Erb's Point, third left intercostal space (left sternal border) for listening to heart function. Others prefer the apex of the heart, fifth left intercostal space (midclavicular line). This iteration and other chest sensor embodiments of the proposed invention facilitate important flexibility in the precise placement of sensors depending on the particular information sought.

The chest or torso is also advantageous for swimmers due to relatively less motion at that location while swimming. This could result in fewer sensor artifacts. Chest mounted displays also facilitate more stable viewing of data in reflective surfaces (i.e. mirrors, holograms, etc.) on the bottom and/or sides of the pool or in the water, itself (holograms), for face-down swimmers. Backstroke swimmers could view data in overhead (above water), mirrors, as they swam underneath. Overhead mirrors are commonly used for backstroke while swimming in-place in ENDLESS POOLS®, MASTER SPAS® Michael Phelps LEGEND®, RIVER POOLS® or other flow-current pool systems (“aquatic treadmills”).

Digital or alphanumeric data would be readable, in proper orientation, by the swimmer in detached reflective surfaces (i.e. mirrors or holograms) because the display attached to the swimmer would show numbers and letters laterally inverted. The reflection process would “reverse” the original image from the point of view of the swimmer for proper interpretation.

Non-alphanumeric, color coded or blinking light coded data could also be readily understood by the swimmer, in real-time, on reflective surfaces, whether laterally inverted or not from the body worn or attached display. Reflective surfaces on long flat substrates (including, but not limited to mirrors) may utilize special optics to improve visibility and/or conspicuity of images reflected back to swimmer's eyes through water. Reflective surfaces may be held securely on pool bottom by weight and/or double-sided suction cups made of chlorine resistant materials and/or other means. Multiple mirror shapes may be used (planar, convex, concave, “banana,” etc.) to improve quality and/or field of view for swimmers in neighboring lanes. And special goggle and/or mirror optics may be used for improved image transmission and viewing through water. Goggles may use enhanced optics to improve visibility and/or conspicuity of alphanumeric images reflected back, now properly oriented, in the mirror image.

This embodiment could also employ additional devices at different locations of the body, including, but not limited to, the back, rib cage, neck, carotid, inner elbow, triceps, etc. These additional display locations, with normally oriented optical output (not laterally inverted), would facilitate direct, simultaneous views of alphanumeric and/or non-alphanumeric (for example, without limitation, pre-programmed color coded or blinking light heart rate zones) optical data, in real-time, for observers (i.e. coaches) on deck.

As stated, the underwater device, on the chest of a face-down swimmer, would show laterally inverted digital optical output or color coded pre-programmed biometric data zones, including but not limited to heart rate. This optical data would be reversed in the mirror image and readable to face-down swimmers when reflected from mirrors on the pool bottom. Non-alphanumerical data would be understandable whether laterally inverted or not.

The devices in this example include, but are not limited to, flexible, comfortable, waterproof, submersible adhesive (using, without limitation, bonding agents and/or nanotechnology and/or other adhesive architecture technology) and/or non-adhesive (i.e. including, but not limited to, compression by elastic and/or other forces) skin strips, patches or wearable gear, as described throughout this APPLICATION. This chest positioned embodiment could be self-contained and/or work with and/or support and/or augment and/or secure other devices (by way of example and not limitation, Garmin®, Polar®, Apple®, Fitbit®, etc., with wrist watch bands detached and/or replacing wrist bands with a chest band.) containing, all the necessary circuitry, hardware, sensors, displays, programmable micro-controllers, processors, lighting display technology and software, etc. This chest positioned embodiment would enable close, fixed, optimally tight, secure, intimate relationships to the skin for high fidelity sensing and display of accurate biometrics including, but not limited to heart rate. This embodiment may also be enhanced in conjunction with the double-sided-adhesive “sensor-bridge” embodiment described herein below.

It should be mentioned that backstroke swimmers (face-up swimmers) could view data directly and/or in above-water mirrors, suspended overhead. Coaches observing backstroke swimmers could simultaneously and directly view displays of biometric optical output including, but not limited to, heart rate, from a device and/or devices mounted on the chest or other advantageous monitoring site on these face-up swimmers.

Of course, as implied above, this embodiment could augment other, more traditional, forms, including a common chest strap but might exhibit some of the disadvantages (including form factor) discussed above.

Another embodiment of the invention includes the coupling of the display with and/or integrated into a swimmer's hand paddle. This embodiment, application and/or configuration of the proposed invention would benefit coaches and their swimmers. An adhesive and/or non-adhesive biometric skin strip or patch device and/or wearable, as described throughout this application, could be coupled with, connected to and/or integrated into a swimmer's hand paddle. Sensors within the device could be positioned at the radial pulse area on the wrist or other advantageous biometric sensing site. Optical output could be displayed in digital, alphanumeric and/or non-alphanumeric and/or color coded and/or blinking format. The swimmer's hand paddle could serve an extension of the display or the actual display screen, itself, integrated into or coupled with the proposed invention (device, apparatus and/or system and/or method). This would provide a larger viewing surface for optical output of biometric data. The entire swimmer's paddle, or a portion of it, could display colors, non-alphanumeric and/or digits and/or alphanumeric data directly to the swimmer (not laterally inverted). Alternatively, the swimmer could see output, properly oriented, in underwater mirrors if laterally inverted at the point of origin from the paddle. Optical output, most likely non-alphanumeric color coded data, would be directly visible to the coach when the swimmer's hand paddle was above or near the surface of the water in the stroke cycle. This embodiment may also facilitate use of other, possibly more accurate, sensor technologies requiring two or more electrode contact points (by way of example and not limitation, EKG, ECG). Please see DRAWINGS, attached.

It is respectfully emphasized, this paddle embodiment is only exemplary and not limiting. For example this iteration might be accomplished with finger rings on both hands, providing multiple, bilateral, points of sensor contact (and color-coded display) for electrical impulse measurement across the heart as used in EKG/ECG type systems (currently the gold standard in biometric medical sensing applications.).

EMBODIMENTS of the invention attached and/or worn under and/or integrated into a bathing cap, hat, helmet, or other head gear, visible to athlete and coach: This embodiment or application of the proposed invention would benefit coaches and their athletes, including, but not limited to, swimmers. Fully enabled adhesive and/or non-adhesive biometric sensing devices, including sensors and displays as described herein would be attached to the skin and/or worn under and/or coupled with and/or integrated with some form of head gear, including a bathing cap, on the forehead, temporal artery or other advantageous biometric sensing site visibly displaying digital, alphanumeric and/or non-alphanumeric and/or color coded and/or blinking data. Head gear, including, but not limited to, a bathing cap, could serve as an extension of the display or a display screen integrated into or coupled with the proposed invention providing a larger view of optical output of biometric data. The entire bathing cap (using various lighting technologies including, but not limited to light emitting diodes, photoluminescence and/or conductive and/or non-conductive polymers), or a portion of it, could display non-alphanumeric colors and/or blinking lights and/or alphanumeric digits, lettered data directly to the swimmer and/or visible to the swimmer in underwater mirrors using the laterally inverted display method previously discussed. Output could be directly visible to the coach. This embodiment could also be applied and have significant value in many other forms of head gear, helmets and contexts. Please consider scuba diver wetsuit hoods. In the scuba diving context it can be important for an accompanying diver (“buddy diver”) to see biometric data of her/his companion diver in real-time. A scuba diver's hood could act as a display screen just as the bathing cap did in this swimmer example. In this way a companion diver, for example, could directly view optical output of heart rate for his/her companion and be alerted to a possible health emergency.

Applications of this embodiment in the context of contact sports, such as football, displaying biometric data indicating head injury or cardiac distress are also obvious.

EMBODIMENT of the invention using the mid-chest as a sensor site and wirelessly transmitting biometric data, via extended NFC (Near Field Communication and/or Near Field Magnetic Induction Communication and/or “NFC+”) and/or Blue Tooth to attachable and/or wearable displays simultaneously visible to the wearer and observers.

This, optionally waterproof, iteration of the invention would include, but not be limited to, one or more biometric (for example and not by way of limitation, heart rate) monitors with one or more sensors attached to and/or worn on and/or inserted into the chest, due to accuracy of heart rate and biometric sensing at that location. Sensor data would be wirelessly transmitted via extended NFC (Near Field Communication and/or Near Field Magnetic Induction Communication and/or “NFC+”) and/or Blue Tooth to wearable displays simultaneously visible to the wearer and observers in real-time. Extended NFC may perform better in an aquatic environment because Blue Tooth signals may attenuate in water.

Attached or worn displays (i.e. without limitation, gear, uniforms and clothing, wristwatches, rings, sleeves, etc.) in this iteration would be capable of showing digital and/or color-coded information. Sensors would be on the sensing-advantageous chest, but displays would be attached to the body and/or integrated into wearables conveniently visible to both the wearer and observers. Wearables might include, but are not limited to helmets, bathing caps, goggles, wrist bands, swim suits, jerseys, socks, shoes, fins, swimming hand paddles, shoulder pads etc.).

EMBODIMENTS employing sensor technologies other than photoplethysmography, including, but not limited to electrocardiography (EKG, ECG).

EKG/ECG fidelity may be enhanced by two or more sufficiently separated sensor electrodes integrated into one, optionally waterproof, flexible biometric adhesive and/or non-adhesive skin strip/patch and/or gear forms of the invention, as described throughout this APPLICATION, or in separate multiple, sufficiently separated, skin patch and/or gear integrated devices. The body-attached and/or worn device and/or devices, including sensor electrodes, could measure varying electrical impulses of the heart to establish heart rate and other biometrics. Optical display, in real-time, directly to the wearer or in reflective surfaces and/or direct views to observers could be accomplished in this iteration of the proposed invention.

Use of ECG/EKG and other sensor technologies (including, but not limited to electrical impulse, and/or pressure sensing and/or sound sensing, deep tissue sensors and/or deep tissue laser sensors) in addition to traditional photoplethysmography in conjunction with optimally close skin sensor connectivity will improve fidelity. This enhanced sensor performance in comfortable form factors will further the stated goals of the invention by using various technologies in different exercises and exercise environments. Biometric electrical impulse sensors and others may prove effective in providing more accurate biometric data than commonly used light sensor technology (photoplethysmography).

This embodiment is not intended to limit the types of sensor technologies that could be advantageously utilized in conjunction with the invention. Sensor technologies synergistic with the proposed invention could include, but are not limited to, electrical impulse (electrocardiography) and/or pressure sensing (oscillometric pulses) and/or sound sensing (phonocardiograph), light sensors (photoplethysmography) and/or chemical sensors and/or quantum sensors and/or diamond nanocrystal sensors and/or nanotechnology based sensors and/or sensors wherein optical fibers are embedded in composite materials (where the material, itself, essentially acts as a sensor and/or display) and/or nanotube sensors and many others.

It should be noted that ECG/EKG, electrical impulse sensors and other sensor technologies may benefit from the use of conductive polymers to enhance sensor readings and/or transmission of electrical signals from the heart through the skin. Conductive polymers may also enhance optical display of data.

SOME LAND BASED EMBODIMENTS with or without a portable mirror may include, but are not limited to, people attaching and/or wearing the proposed invention while exercising on machines not equipped with data screens (i.e. treadmills, rowing machines, skiing machines, stationary bikes, etc.). These exercisers could view, either directly and/or indirectly in reflective surfaces, accurate biometric and other digital, alphanumeric and/or non-alphanumeric color coded or blinking light coded information, such as heart rate, taken from the accuracy-advantageous chest (sternum, solar plexus areas etc.), carotid, ankle, femoral artery, radial pulse wrist, inner elbow and/or many other locations on the body. The invention facilitates use of numerous biometric sensing locations, well beyond traditional sites, to provide accurate data due to the close, high quality contact provided by epidermal strips/patches/gear as described throughout this APPLICATION. Optical output of biometric data could then be viewed by people on exercise machines, either directly (without mirrors) and/or indirectly, in real-time, without interrupting exercise movements using a lightweight portable mirror. Digital, alphanumeric output would be initially displayed (by way of example, without limitation, from the upper chest or lower neck) in a laterally inverted manner. The image would be “reversed” and correctly viewed by the observer/wearer when reflected back in a mirror. Pre-programmed color coded biometric zones (non-alphanumeric data) would easily be read by the exercising person, either by direct view and/or in the reflection surface. Coaches, trainers and fitness class teachers could conveniently and simultaneously view color coded optical data and/or view alphanumeric data from a non-laterally inverted data display.

SOME LAND BASED EMBODIMENTS in exercise rooms with and/or without wall mounted mirrors include, but are not limited to, exercises that take place in rooms with mirrors (i.e. aerobics classes, cycling classes, skipping rope, jumping jacks, dancing, etc.). These exercisers could immediately view, while in motion, accurate biometric and other digital, alphanumeric and/or non-alphanumeric data (i.e. color coded or blinking light coded information), in real-time, including, but not limited to, heart rate taken from the accuracy-advantageous chest area or elsewhere on the body. This data would easily be viewed in wall mirrors, typically available in exercise rooms, in real-time, without disrupting exercise. Digital data (i.e. 7 segment display or other display technology) could be viewed by the exercising people in proper orientation because the output display attached to and/or worn by the exerciser would be laterally inverted and reversed upon reflection in the mirror. If coaches or class instructors also wanted to directly view properly oriented alphanumeric data from exercisers then additional body-attached and/or worn displays with normal (non-laterally inverted) output could be provided. Alternatively, one display could provide both normal and laterally inverted optical readouts. This would facilitate properly oriented direct and reflected views from one screen for exercisers and teachers facing them. Non-alphanumeric, color coded or blinking light coded data would be readily understandable whether read directly or in a mirror. For example, the color coded light output by the device may have different wavelengths depending on the readings taken by the sensor. Instructors, for example in cycling classes, would benefit from seeing data from a normally oriented display on the rider or a display on the rider that alternates between laterally inverted and regular display of optical biometric output. Instructors need this information to assess the various fatigue levels of students, especially in larger classes.

Another embodiment of the invention includes a coupling device (or “sensor-bridge”) using lenses, discs, patches and/or strips with double-sided adhesive qualities, with or without integrated electronics and/or optics, to improve sensing accuracy of wearables commercially available in the marketplace.

This double-sided-adhesive biometric “sensor-bridge” includes a substrate with first and second sides that are generally parallel to each other. As stated above, the adhesive covers both the first and second sides. The “sensor-bridge” attaches to, augments and works in conjunction with commercially available heart rate monitoring wearables. It may be conceptualized as the filling of an “Oreo Cookie.” The sensor side on the back of a wrist wearable (i.e., without limitation, an Apple® Watch) is the top cookie layer. The skin of the user is the bottom cookie layer. In-between is the sensor-fidelity-enhancing device or “filling.” Iterations of this “sensor-bridge” device (i.e., the center or “filling” in our analogy) could have a wide range of pliability from flexible to rigid.

This supplemental coupling device would have appropriate adhesive qualities on both of the first and second sides. Adhesion methods may be different on the skin side than on the sensor side, possibly requiring two different adhesives. It would serve as a connecting data transmission bridge or bus between skin and sensors on commercially available wearables, such as wristwatch type monitoring devices. It would fill and secure the gap between the epidermis and sensors. This embodiment would create a close, optimally intimate skin-sensor contact. This would facilitate high fidelity and/or magnified and/or enhanced and/or beneficially filtered pass-through of outgoing (emitter) and returning (rebounding) biometric sensor light to improve accuracy of biometric data readings. This could improve the accuracy of commercially available biometric monitors and wearables and/or devices including, but not limited to, current models by Garmin®, Polar®, Apple®, Fitbit®, etc.

This embodiment employs patches or strips with adhesion qualities on both sides so it can stick to skin on one side and the back of wrist-watch type wearables on the other side, where sensors are located. These double-sided adhesive devices may use a variety of adhesive technologies on the skin side of the device that may or may not be different than those used on the sensor side. They may adhere using chemical and/or compressive and/or mechanical and/or nanotechnology and/or other adhesion methods, with or without “glue.” The devices could, optionally, be waterproof, patches, strips, discs or lenses, with or without integrated electronics and/or optics and/or signal enhancing and/or conducting nanotechnology and/or nanotubes and/or polarizers and/or filters, light contrasting methods, special lenses, and/or amplifiers, magnifiers, and/or prisms and/or light differentiators to improve the efficacy of sensor readings through the skin, including, but not limited to light sensors predominately used in wearables available in the marketplace today. These double-sided adhesive devices would create a stable, secure, fixed, constant link, filling the gap between sensors and skin. This embodiment creates a close, appropriately tight, sensor-to-skin contact providing a conduit or bus through which high fidelity biometric data can be harvested. Outbound light from light sensors (or other sensing modalities, by way of example, but not limitation, electrical impulse) may be magnified, amplified, polarized, magnetized, filtered or otherwise enhanced as it passes through the gap-filling device into the body and/or as it rebounds back through to sensors. This transdermal bridge would improve the accuracy of biometric data sensed inside the body of active exercisers and conveyed sensors in commercially available wearable monitoring devices including (by way of example, but not limited to) wrist-watch type products by Garmin®, Polar®, Apple®, Fitbit®, etc. Metaphorically this embodiment is intended to help existing wearable sensors in the marketplace “see” better into the body, during active exercise, and report that “view” back to sensors with greater accuracy.

Wearable monitors, especially wrist wearables, available to the general public that attempt to provide heart rate data and other biometrics in real-time, are very inaccurate while the wearer is in motion (see, American College of Cardiology, Marc Gillinov, MD, Cleveland Clinic, Mar. 8, 2017). The main reason for this inaccuracy could well be the lack of a secure, fixed, intimate connection between the skin of the wearer and sensors on those wearables, especially during physical activity. Most, if not all available wearable monitors on the market use light sensor technology. This embodiment would improve the fidelity of readings from light sensors and/or other sensor methods on existing products by building a double-sided adhesive, seamless, fixed, connective communication “bridge” between skin and sensors.

Unstable skin-sensor pressure contact and/or gaps and/or interposition of water or sweat in gaps between sensors and the epidermis and/or varying skin perfusion rates during activity distort sensor readings for multiple reasons. Those reasons include, but are not limited to, reduced detectability of internal biometrics with widening gaps and failure to stabilize skin perfusion rates as sensor-to-skin contact ebbs and flows during exercise. The contact between sensor and skin needs to be stable and “just right” (not too loose, not too tight). If too loose, sensors may be less effective. If too tight, excessive pressure may impact blood flow and/or skin perfusion rates. These and other data sensing distortion problems in commercially available heart rate monitoring devices could be improved by this embodiment.—Just as a physician skillfully holds a stethoscope to the chest to amplify and listen to the heart beat, sensors must also be skillfully held to the skin.

This embodiment may be active (powered) or passive (not powered). If the embodiment requires power it could be charged inductively from sources affiliated and/or not affiliated with existing wearable monitoring devices in the marketplace that are charged inductively. Also, there may be readily available methods of powering this embodiment directly from the wearables to which it attaches. There are several wrist wearable heart rate monitors in the marketplace that are currently enabled with Bluetooth as well as NFC (Near Field Communication) and/or NFMIC (Near Field Magnetic Inductive Communication) chips (i.e. without limitation, Apple®, etc.).

This double-sided sensor-to-skin “bridging” embodiment would work in conjunction with and/or be programmed through dedicated “Apps” on wristwatch type wearables (i.e. Garmin®, Polar®, Apple®, Fitbit®) to display the more accurately sensed data it facilitates in color-coded heart rate zones (and/or other biometrics). This more accurate, color-coded optical output could be viewed, in real-time, by exercisers (i.e. swimmers) and observers (i.e. coaches).

This embodiment also contemplates product specific, dedicated, double-sided specialty adhesive cases, frames, gaskets, patches or strips to secure a close skin-to-sensor connection. Adhesive technologies (including, but not limited to waterproof bio-adhesives) could be employed on all, or portions, of one or both sides of these product-dedicated cases and/or gaskets, strips or patches to frame in and hold “sensor-bridge” devices in place. This would facilitate affixing of sensors optimally close to the skin at the best biometric data harvesting locations for a given exercise.

For example, wristwatch bands could be removed from wristwatch-type wearable heart rate monitoring devices commonly found in the marketplace. Then the manufacturer's device, minus the watch band, could be secured on the chest using a double-sided, adhesive “sensor-bridge.”

This embodiment may include sensor windows carved out of the double-sided-adhesive patches and/or strips and/or cases and/or frames. Adhesively contacting portions of the double-sided adhesive device could optimally and securely hold commercially available heart rate monitor sensors in place while leaving sensor light pathways unaffected.

Product dedicated, double-sided adhesive and/or compressive gaskets, patches and/or strips and/or cases with adhesive technologies (including, but not limited to, bio-adhesives) could be used to hold a manufacturer's monitor in place for optimal sensing fidelity at optimal sensing locations.

It may or may not be advantageous to use adhesive methods on sensor light pathways. This variation, with open sensor windows, could facilitate secure, fixed, efficiently close skin-to-sensor contact while avoiding possible obstruction of light sensor paths by adhesion methods. However, it should be emphasized that sensor-bridge embodiments containing clear lenses and/or polarized lenses, enhanced optics, filters and/or electronics (as described herein) may or may not be used between skin and sensors with a given manufacturer's monitoring device and/or on a given individual's skin.

There are many variations on this double-sided sensor-bridge embodiment. It should not be construed as being limited to light sensors. By way of example, and not limitation, conductive polymers (including, without limitation, graphene) could be used to enhance electrical impulse sensor communication and fidelity in EKG/ECG type sensors.

PREFERRED, NON-ADHESIVE EMBODIMENTS USING COMPRESSION SLEEVES made with elastomers, rubber-like materials (by way of example and not limitation, silicone or elastic, stretchy, conformal materials) capable of displaying color-coded data from integrated or separate bio-sensors including, but not limited to heart rate.

These examples of the invention would primarily rely on compressive elastic forces to provide close, secure, intimate contact of sensors to the skin on a multitude of advantageous bio-metric sensing and convenient display sites on the body, facilitating accurate bio-metric readings. All necessary electronics, including but not limited to sensors, displays, lighting, electronics, micro-processors, programmable hardware, micro-controllers, could be integrated, enveloped, layered and/or encapsulated and/or molded into an optionally waterproof, submersible, silicone-type material. Said material would be capable of illumination (using various lighting technologies including, but not limited to light emitting diodes, organic light emitting diodes, photoluminescence and/or conductive polymers, non-conductive polymers, nanotubes with optical properties, etc.) to display programmed, non-alphanumeric, color-coded and/or blinking light bio-metric zones (including, but not limited to heart rate). These embodiments could use part or all of the compression sleeves as displays visible to the wearer and observers, such as coaches, in real-time, during activity. Elastic forces could provide the close, conformal skin-sensor contact essential to high fidelity bio-metric readings including, but not limited to, heart rate. Please see DRAWINGS, attached.

In uses where waterproofing was desired inductive charging and/or other methods of wireless charging and programming [including, but not limited to programming by “Internet of Things” (JOT) Bluetooth®, NFC) from computers or handheld devices with or without dedicated “Apps”] would be preferable to limit potential entry points for water into the electronics. In dryland applications charging through ports would be acceptable.

Although adhesive surfaces may not be necessary because compressive elastic forces would primarily secure the essential skin-to-sensor contact, this preferred embodiment is not limited to non-adhesive technologies. It could also employ adhesive bonding methods or nanotechnology bonding architectures. However, it is separately stated here as a “non-adhesive embodiment” to emphasize that such “sticky” technologies may not be necessary in this and many applications. There may be benefits to using non-adhesive technologies on human skin.

There are multiple variations of this “compression sleeve” embodiment that could enhance sensor-to-skin contact for more accurate biometric readings. They include, but are not limited to:

Product-dedicated windows (open or covered with a see-through materials) in compression sleeves could facilitate removal of bands from wrist watch or chest type monitors currently in the marketplace. Bands could be replaced by sleeves that may provide better all-around compression forces and, possibly, form factors. Sleeves could also allow positioning of commercially available biometrics monitors with or without attached “sensor-bridges” (as described above) at multiple locations on the body. This could provide opportunities for improved sensing and display of data to wearers and observers. Optionally waterproof, comfortable sleeves, with window openings could be used to hold commercially available monitors between skin and sensors with or without a “sensor-bridge” (as described above) using elastomeric compression forces and/or adhesive technologies (including, but not limited to bio-adhesives) as described in the “sensor-bridge” embodiment, above. “Sensor-bridges” containing enhanced optics and/or electronics (as described above) may or may not be interposed between skin and sensors in the manufacturer's monitoring device.

PREFERRED, NON-ADHESIVE EMBODIMENTS USING GOGGLE AND/OR MASK FRAMES AND/OR GASKETS AND/OR STRAPS made of clear and/or translucent elastomers (by way of example and not limitation, silicone or rubber-like, stretchy, conformal materials) capable of displaying color-coded data from self-contained bio-sensors including, but not limited to heart rate.

This embodiment of the invention would primarily rely on compressive elastic forces to provide close, secure, intimate contact of sensors to the skin of the face and/or eye orbit areas (or other advantageous biometric sensing site around the eyes, on the face and/or head and/or neck) for accurate biometric readings. This iteration takes advantage of the elastic, compressive forces found in swimming goggles, masks gaskets, frames and connecting head straps. Conductive and/or non-conductive polymers and/or elastomers may be used in this embodiment. Constituent materials could include, but not be limited to, clear and/or translucent and/or any stretchy materials facilitating lighted optical display. Although frequently made of non-conductive polymers, there may be advantages to constructing all or part of this embodiment out of conductive elastomers and/or polymers (i.e., without limitation, intrinsically conducting polymers, such as layered or molded graphene or G-elastomers, and/or conductive polymers with metallic conductivity or non-metallic conductivity, and/or semiconductors). These conductive materials could open the door to using a variety of sensor modalities in addition to light sensors (including, but not limited to electrical impulse, pressure sensing, sound sensing, deep tissue sensors and/or deep tissue laser sensors, and/or other existing and/or future sensor methods, etc.). All necessary electronics, including but not limited to, sensors, displays, lighting, electronics, circuitry, micro-processors, programmable hardware, micro-controllers, batteries, inductive charging hardware could be hermetically sealed, enveloped, potted, layered, laminated and/or encapsulated and contained in waterproof, submersible, silicone-type materials. Goggle/mask materials would include, but not be limited to, elastomers and/or polymers (by way of example and not limitation, silicone and/or silicone-like, rubber-like, stretchy polymers and/or materials) conformable to the contours of the human face. The same elastic, compressive forces that swimming goggles, masks and straps use to provide a water-tight seal around the eyes would also facilitate the close, intimate, appropriately skin-tight sensor bond so essential to high fidelity monitoring of heart rate and other biometrics. Accurately sensed data, including but not limited to heart rate, would then be displayed in pre-programmed, colored lights or blinking lights. Goggle/mask materials could be clear or translucent (i.e. see-through, semi-transparent or milky colored through use of diffuser) and/or otherwise capable of illumination (using various lighting technologies including, but not limited to light emitting diodes, organic light emitting diodes, luminescence, photoluminescence and/or conductive polymers, non-conductive polymers, nanotechnology, nanotubes with optical properties, etc.) to sense and display programmed, non-alphanumeric, color-coded and/or blinking light bio-metric zones (including, but not limited to heart rate). These embodiments could use part or all of the goggle gasket frame and/or straps to simultaneously display accurate optical output to both athlete and coach in real-time while an athlete, such as a swimmer, was in motion.

In uses, such as swimming goggles, where waterproofing was desired inductive charging and/or other methods of wireless charging and programming [including, but not limited to programming by “Internet of Things” (JOT) Bluetooth, NFC) from computers or handheld devices with or without dedicated “Apps”] would be preferable to limit potential entry points for water into the electronics. In dryland applications charging through ports would be acceptable. Also, it may be preferable to wholly contain electronics in frames, straps, gaskets made of materials, as described above to enable replacement of scratched lenses new lenses for changes in eye prescriptions of the wearer, if applicable. This is not intended to limit use of lenses as displays or sensors in this or any embodiment. Lenses could be edge-lit without containing any electronics and thereby act as a light display if this is determined to be necessary in lieu of or in addition to rim and strap lighting. Lenses may be a source of sensors harvesting bio-metric data directly from the eye or surrounding areas.

This embodiment could be used in many other forms of eye ware and contexts including, but not limited to, scuba diving masks, ski masks, basketball face, nose and eye protection masks/goggles, paddle ball safety goggles, eye contacts, etc. Of course, this iteration could also be made in a non-waterproof version for land based use.

Although adhesive surfaces may not be necessary because compressive elastic forces could secure the essential skin-to-sensor contact, this preferred embodiment is not limited to non-adhesive technologies. It could also employ adhesive bonding methods or nanotechnology bonding architectures (with or without “glue.”). However, it is separately stated here as a “non-adhesive embodiment” to emphasize that such “sticky” technologies may not be necessary in this and many other applications where appropriate compressive forces or other technologies may maintain excellent skin-sensor contact during activity.

Again, it is respectfully emphasized, the above embodiments are not intended to limit applications of the invention in any way. Swimming contexts for the invention have been emphasized because they are of great interest to the inventors. Moreover, the aquatic environment is harsh and challenging. If the invention is effective in aquatic settings it bodes well for its multiple, widespread uses in many different environments.

The proposed invention, whether construed as a device, apparatus, system and/or method, as described throughout this APPLICATION will enable accurate sensing and convenient display of important metrics in real-time to an active person and observers (such as coaches) in a variety of circumstances. The wearer would not have to peer at small, awkwardly located, readout screens and disrupt exercise movements. Coaches would not be forced to look at hand-held devices for real-time information or pour over archived data after training sessions with multiple athletes. Coaches would directly and immediately see accurate, optical biometric data, including but not limited to heart rate, from athletes in motion. The disadvantages described in the BACKGROUND section, above, could be avoided and the goals of the invention achieved.

This APPLICATION FOR UNITED STATES UTILITY PATENT is not intended to limit the proposed invention in its embodiments, applications, designs, iterations, hardware, software, programmability and/or uses in combination and/or integration with other possible affiliated and/or supportive components and/or technologies, [i.e. including, but not limited to displays; sensor technologies including but not limited to electrical impulse (electrocardiography) and/or pressure sensing (oscillometric pulses) and/or sound sensing (phonocardiograph) and/or chemical sensors, dielectric sensors, sensor inks, radio frequency identification (RFID) tags, quantum sensors and/or diamond nanocrystal sensors and/or nanotechnology based sensors and/or sensors wherein optical fibers are embedded in composite materials (where the material, itself, essentially acts as a sensor and/or display) and/or nanotube sensors; micro-controllers, processors, integrated chips, applied specific integrated chips, light emitting diodes, optics, lenses, enlargers, image magnifiers, image projectors, EKG/ECG electrodes, accelerometers, inductive chargers, illuminated and/or luminescent, photoluminescent displays, reflective surfaces, eyewear, garments, so-called “smart textiles”, electronics printed onto the skin, circuitry and electronics from a 3D printer and attached to the skin, electronics tattooed and/or inked onto/into the skin (possibly making the skin interactive or interface with sensors and internal biometrics), apparel, gloves, bands, sleeves, leggings, swimsuits, wristwatch devices, adhesives, skin bonding and/or attachment nanotechnologies and/or architectures, and/or waterproofing technologies, waterproof adhesive technologies, micro skin interface stitching, electronic deep tissue sensors, goggles and/or goggle straps, ear lobe clip sensors, waterproofing techniques such as sealing by layering and/or flooding and/or encapsulation, below the skin chip and/or sensor insertion, hologram projection display, goggles lenses and/or contact lenses with sensors and/or displays, micro-controllers and other hardware and software, algorithms reducing artifact distortions during activity, any and/or all symbiotic, supportive and/or augmenting and/or synergistic technologies etc.].

The invention could be disposable or long lasting. It could be provided in embodiments that are not waterproof or submersible if land based uses did not require it. Moreover, the invention could, for example, be combined and/or coupled with other forms of communicating output, rather than just optical output, including, but not limited to, vibration or sound. By way of example only, and not limitation, the proposed body worn and/or attached device could also vibrate or beep alerting an athlete to start or stop exercise as a function of time, distance, speed, acceleration, number of repetitions, time intervals, heart rate, blood pressure and other biometrics and/or metrics. The invention could also display time elapsed for a given distance traversed. This would be valuable for traditional interval training and the relatively new swimming training method called “Ultra Short Race Pace Training” (USRPT).

This APPLICATION is not intended to limit uses of the proposed invention in any way whether standing on its own terms and/or in novel, synergistic, symbiotic use with other inventions and/or technologies.

The present invention may be construed as a device and/or apparatus and/or system and/or method and is not limited to the embodiments described above, but encompasses any and all embodiments expressed and/or implied and/or inferable. The claims, below should be construed as both dependent and independent claims.

We claim:

1. A biometrics device for measuring a biometric quantity of a body of a user in motion, said biometric device comprising:
a sensor to measure the biometric quantity of the user and to create a sensed biometric signal;
a coupling device to couple said sensor to the body of the user; and
an emitter electrically connected to said sensor for receiving said sensed biometric signal and emitting an output signal to be received by the user such that the user can understand the biometric quantity as the user continues the motion.

2. A biometrics device as set forth in claim 1 wherein said emitter includes a light source to emit light out and away from said emitter.

3. A biometrics device as set forth in claim 2 wherein said light source emits light having a specific wavelength based on said sensed biometric signal.

4. A biometrics device as set forth in claim 3 including a diffuser operatively connected to said light source for dispersing the light in a plurality of directions.

5. A biometrics device as set forth in claim 4 wherein said light source emits the light in an amplitude visible at a distance 50 meters from the user.

6. A biometrics device as set forth in claim 1 wherein said coupling device includes a transparent substrate having first and second surfaces extending parallel to each other.

7. A biometrics device as set forth in claim 6 wherein said coupling device includes first and second layers of adhesive covering each of said first and second surfaces, respectively.

8. A biometrics device as set forth in claim 7 wherein said first and second layers of adhesive are fabricated from different materials.

9. A biometrics device as set forth in claim 6 wherein said substrate defines a hole extending therethrough.

10. A biometrics device as set forth in claim 9 wherein said substrate includes a lens extending through said hole to focus the light passing therethrough.

11. A biometrics assembly for measuring a biometric quantity of a body of a user in motion, said biometrics assembly comprising:
a biometrics device having first sensor to measure the biometric quantity of the user and to create a sensed biometric signal, a coupling device to couple said sensor to the body of the user, an emitter electrically connected to said sensor for receiving said sensed biometric signal and emitting an output signal to be received by the user such that the user can understand the biometric quantity as the user continues the motion, a receiver for receiving an on/off signal to remotely turn said emitter on and off; and
a transmitter remote from said biometrics device having an illumination switch for turning said emitter on and off.

12. A biometrics assembly as set forth in claim 11 wherein said emitter includes a light source to emit light out and away from said emitter.

13. A biometrics assembly as set forth in claim 12 wherein said light source emits light having a specific wavelength based on said sensed biometric signal.

14. A biometrics assembly as set forth in claim 13 including a diffuser operatively connected to said light source for dispersing the light in a plurality of directions.

15. A biometrics assembly as set forth in claim 14 wherein said light source emits the light in an amplitude visible at a distance 50 meters from the user.

16. A biometrics assembly for measuring a biometric quantity of a body of a user in motion, said biometrics assembly comprising:
a biometrics device having first sensor to measure the biometric quantity of the user and to create a sensed biometric signal, a coupling device to couple said sensor to the body of the user, an emitter electrically connected to said sensor for receiving said sensed biometric signal and emitting an output signal to be received by the user such that the user can understand the biometric quantity as the user continues the motion; and
a substrate with a reflective surface spaced apart from the user to reflect the output signal back toward the user such that the output signal is visible to the user without the user looking directly at said emitter of said biometrics device.
